(19) United States
(12) Patent Application Publication
(10) Pub. No.: US 2004/0082766 A1
(43) Pub. Date: Apr. 29, 2004

(54) Method of purifying biological substances, kit for purifying biological substances, and system for analyzing biological substances

(75) Inventors: Hiroko Hanzawa, Tokorozawa (JP); Kenko Uchida, Setagaya (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/448,383

(22) Filed: May 30, 2003

(30) Foreign Application Priority Data
Oct. 29, 2002 (JP) 2002-313620

(51) Int. Cl. C07K 1/00; C07H 21/04

(52) U.S. Cl. 530/413; 536/25.4

(57) ABSTRACT

A method of purifying biological substances, a kit for biological substance purification, and a system for analyzing a biological substance are provided. In one example, a method of purifying biological substances is provided. The method comprises preparing a carrier species having a surface covered with a noble metal, wherein a surface of the noble metal is surface-treated for binding a capturing molecule species to a biomolecular species; binding the capturing molecule species to the surface of the noble metal to create one or more carriers; feeding a sample containing the biomolecular species to a container containing the one or more carriers; causing the biomolecular species to bind to the capturing molecule species; feeding a solution for one of dissociating or eluting the biomolecular species bound to the capturing molecule species on the one or more the carriers therefrom; and recovering the biomolecular species.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention generally relates to purifying biological substances. More particularly, the invention relates to a surface-treated carrier for purifying a specific biological substance, to a method of purifying a biological substance such as a protein, nucleic acid, antibody or hormone, to a kit for carrying out such a method, and to an analyzing system for analyzing a substance purified by such a method or kit.

2. Discussion of Background

A biological phenomenon consists in a series of orderly regulated biological reactions. Not only in the field of biological sciences but also in the field of medical applications. For instance, it is an important subject to recover respective components involved in the interaction between biological molecules in each elementary process in that series of reactions and analyze the process of such interaction.

In recovering a specific biological substance, that biological method which utilizes the specific affinity between molecular species is in general use. Accordingly, immunoprecipitation and affinity chromatography using an affinity adsorption carrier have so far been used for recovering a specific intracellular biological substance. The conventional affinity adsorption carrier comprises carrier or support particles having a particle size of about scores to hundreds of micrometers with a ligand bound thereto via a spacer having an appropriate length, and a porous crosslinked polysaccharide matrix, typically an agarose gel carrier, is most often used as the support or carrier. Such a form of affinity adsorption carrier is used batchwise or packed in a column for recovering a biological substance specifically binding to the immobilized ligand.

Attempts have been made for a relatively long time to provide a substrate or carrier with a certain function by immobilizing some or other molecular species on the surface thereof. Owing to the discovery, by Allara et al., of the fact that a thiol group-containing alkyl chain (alkanethiol) can react with gold to form a direct covalent bond, it has become possible to immobilize a ligand on the gold surface with a high density and a high level of orientation by introducing a functional group into the terminus of the alkyl chain (cf. e.g. Non-patent Document 1: Nuzzo, R G, Allara, D L: J. Am. Chem. Soc., 1983, vol. 105, pp. 44-81). By binding, based on this principle, a ligand to the gold surface of a substrate via an alkyl chain as the spacer, it is possible to detect the interaction with a specific molecule as a change in electric signal, in diffractive index, or in frequency (number of vibrations). Thus, for example, as disclosed in JP-A No. 326193/1999, particles of polystyrene or the like are disposed on a flat substrate, followed by vapor phase deposition of gold to a thickness of 0.005 to 0.5 μm, whereby the particle surface is partly covered with gold. An arbitrary ligand is immobilized on this gold surface by the method mentioned above for detecting or assaying the interaction with a specific molecule (Patent Document 1: JP-A No. 326193/1999).

The immunoprecipitation or affinity chromatography using the conventional affinity adsorption carriers is low in the efficiency of ligand-biological substance binding and requires a long period of time for the binding. Furthermore, the proportion of the biological substance lost during the series of procedures is high, and the recovery efficiency and the degree of purification are low. Therefore, especially when the content of the target biological substance in the starting material is very low, the starting material is required in very large amounts.

In the current state of the art, it is also difficult to attempt to analyze, by means of a mass spectrometer, a very small amount of a biomolecular species obtained by using a conventional affinity adsorption carrier. The prior technology using particles comprises arranging particles whose surface has been partly vapor deposited with gold and immobilizing a ligand on the gold surface (e.g. Patent Document 1). However, these particles are intended for use in detecting or assaying the interaction of the ligand with a specific substance but are not intended for recovering such specific substance.

### SUMMARY OF THE INVENTION

Broadly speaking, the present invention provides a method of purifying biological substances, a kit for biological substance purification, and a system for analyzing a biological substance. It should be appreciated that the present invention can be implemented in numerous ways, including as a process, an apparatus, a system, a device or a method. Several inventive embodiments of the present invention are described below.

In one embodiment, a method of purifying biological substances is provided. The method comprises preparing a carrier species having a surface covered with a noble metal, wherein a surface of the noble metal is surface-treated for binding a capturing molecule species to a biomolecular species; binding the capturing molecule species to the surface of the noble metal to create one or more carriers; feeding a sample containing the biomolecular species to a container containing the one or more carriers; causing the biomolecular species to bind to the capturing molecule species; feeding a solution for one of dissociating or eluting the biomolecular species bound to the capturing molecule species on the one or more the carriers therefrom; and recovering the biomolecular species.

In another embodiment, a kit for biological substance purification is provided. The kit comprises one or more carriers, each covered on a whole surface of each carrier with a noble metal, wherein a surface of the noble metal is surface-treated in order to bind a capturing molecule species to a biomolecular species of each of the one or more species, wherein the capturing molecule species is configured to recover the biomolecular species.

In still another embodiment, a system for analyzing a biological substance is provided. The system comprises a column or tube containing one or more carriers, wherein each carrier is covered with a noble metal having a magnetic material with the noble metal, wherein a capturing molecule species is bound to a surface of each carrier; an analyzing section connected to the column or tube; a transfer device for transferring the one or more carriers within the column or tube; and a voltage application device configured to guide a biomolecular species to a vicinity of the one or more carriers, wherein the column or tube is fed with a sample containing the biomolecular species for binding of the biomolecular species to the capturing molecule species, wherein the column or tube is fed with a solution for dissociation or elution of the biomolecular species bound to the capturing molecule species on the one or more carriers therefrom to thereby recover the biomolecular species, wherein the analyzing section is configured to analyze the biomolecular species that is recovered.

The invention encompasses other embodiments of a system, a method, and an apparatus, which are configured as set forth above and with other features and alternatives.

### BRIEF DESCRIPTION OF THE DRAWINGS

The present invention will be readily understood by the following detailed description in conjunction with the accompanying drawings. To facilitate this description, like reference numerals designate like structural elements.

FIG. 1 is a schematic illustration, in cross section, of a gold particle used in the practice of the invention;

FIG. 2 is a schematic illustration, in cross section, of a carrier for biomolecule purification according to the invention;

FIG. 3 is an illustration of certain typical spacers capable of binding to gold particles as used in the practice of the invention;

FIGS. 4A-1 is a schematic illustration of a device for increasing the efficiency of binding between a ligand and a specific biological substance by passing an electric current through a buffer solution containing a carrier for biomolecule purification according to the invention, wherein particles move under the influence of a magnetic field without passage of any electric current;

FIGS. 4A-2 is a schematic illustration of a device for increasing the efficiency of binding between a ligand and a specific biological substance by passing an electric current through a buffer solution containing a carrier for biomolecule purification according to the invention, wherein particles move under the influence of a magnetic field while an electric current is passed through the buffer solution;

FIGS. 4B-1 is a schematic illustration of a device for increasing the efficiency of binding between a ligand and a specific biological substance by passing an electric current through a buffer solution containing a carrier for biomolecule purification according to the invention, wherein particles move under the influence of gravity without passage of any electric current;

FIGS. 4B-2 is a schematic illustration of a device for increasing the efficiency of binding between a ligand and a specific biological substance by passing an electric current through a buffer solution containing a carrier for biomolecule purification according to the invention, wherein particles move under the influence of gravity while an electric current is passed through the buffer solution;

FIG. 5A shows the results of western blotting of eluted fractions with a glycine solution and labeled with an anti-mouse IgG antibody;

FIG. 5B shows the results of eluted fractions with an SDS-containing solution, of IgG remaining on the particles after the above glycine solution elution;

FIG. 6 is a schematic illustration of a carrier for biomolecule purification according to the invention as packed in a column;

FIG. 7 is a schematic illustration of a method of protein separation using a carrier for biomolecule purification according to the invention;

FIG. 8 is a graphic representation of the relationship between the ratio of binding between a ligand and a biological substance and the time of binding reaction with the ligand under application of a voltage;

FIG. 9 is a schematic illustration of a method of transferring a sample prepared by using a carrier for biomolecule purification according to the invention to a mass spectrometer;

FIG. 10 is a schematic illustration of a method of subjecting a sample prepared by using a carrier for biomolecule purification according to the invention to high-performance liquid chromatography for concentration of the sample after separation; and

FIG. 11 is a schematic illustration of a micro reaction tube for use in the practice of the invention.

### DESCRIPTION OF THE PREFERRED EMBODIMENTS

An invention for a method of purifying biological substances, a kit for biological substance purification, and a system for analyzing a biological substance is disclosed. Numerous specific details are set forth in order to provide a thorough understanding of the present invention. It will be understood, however, to one skilled in the art, that the present invention may be practiced without some or all of these specific details.

General Overview

According to the present invention, a ligand is immobilized on a carrier or support whose surface is covered with a noble metal, to thereby capture a biomolecular species and thus recover and purify the biomolecular species.

The carrier of the invention has a surface covered with a noble metal. The noble metal includes, within the meaning thereof, gold, platinum, silver and copper. Unlike the conventional affinity adsorption carriers having an agarose network structure, the carrier of the invention is smoothly covered with a noble metal and, therefore, can reduce the loss of a biomolecular species and increase the efficiency of recovery and of purification.

The carrier to be used in accordance with the invention is not less than 0.1 μm but not more than 10 μm in diameter. When the diameter is less than 0.1 μm, the particles are so small that the experimental procedure becomes difficult to perform and, when it exceeds 10 μm, the use of the carrier with a biomolecular species trapped or captured thereon for further analysis, in particular, may be restricted. Accordingly, the above range is adequate for the carrier of the present invention.

In cases where a carrier greater in specific gravity than 1.0 is used as the carrier of the invention, the centrifugation procedure can be carried out in a shorter time as compared with the conventional carries. Furthermore, it also becomes easy to repeat the separation and washing steps in the process of immunoprecipitation, with the result that the nonspecific adsorption can be reduced.

In cases where the carrier of the invention has a magnetic material layer introduced therein, the carrier can be manipulated magnetically. In recovering a specific biomolecular species binding to the ligand from a mixture using this carrier, the carrier with the biomolecular species trapped on the ligand thereof can be separated magnetically by applying a magnetic field after mixing up the carrier with the mixture. Further, since magnetic separation can realize more trustworthy separation in a shorter period of time as compared with centrifugal separation, it is possible to remove contaminants and, as a result, reduce the non-specific adsorption with ease by repeating magnetic separation and washing/rinsing.

The ligand to be bound to the carrier according to the invention is bound to the carrier surface covered with a noble metal either directly or via a spacer having an appropriate length. When a thiol group-containing alkyl chain is covalently bonded as the spacer with a high density onto the carrier surface covered with a noble metal, the ligand can be bound to the tip of the spacer alkyl chain with a high density and a high degree of orientation, so that the efficiency of biomolecule binding can be increased. For some ligand substances, when they are directly bound to the particle surface, there may arise the possibility of their binding with a specific biological substance being prevented by steric hindrance. In such a case, an arbitrary spacer having an appropriate length can be selected and used for ligand binding to the carrier surface. Recommended as the spacer species are alkanethiols having an amino, carboxyl or hydroxyl group which can be chemically modified in various ways.

The substance to serve as the ligand in the practice of the invention is desirably one interacting with or showing affinity for another substance or other substances intracellularly and/or extracellularly in the living organism. As examples of such, there may be mentioned antigens and antibodies, enzymes, nucleic acids having a complementary sequence, receptors occurring on the cell membrane surface, active sites thereof and biological substances reactive therewith, sugars, and glycoproteins. Any appropriate one can be selected arbitrarily from among these according to the intended object. In cases where the carrier of the invention has electric conductivity, voltage application in a certain direction following addition of the carrier to a biomolecule-containing sample can result in local concentration of the biomolecular species and thus in an increase in the efficiency of trapping the same on the ligand. Further, by allowing the ligand to capture the biomolecule, removing nonspecifically adsorbed substances by washing and applying a voltage in a certain direction, it becomes possible to separate and recover the biomolecule from the ligand with high efficiency.

The present invention further provides a system for analyzing biological substances which system comprises recovering a biomolecular species using the carrier and analyzing the biomolecular species thus recovered. As the analyzing section, a mass spectrometer or a liquid chromatograph, for instance, is used.

The use of the carrier of the invention makes it possible to recover and purify biological substances in a simple and easy manner and without fail. The use of the carrier of the invention further makes it possible not only to separate and recover specific biological substances but also to comprehensively search for and detect unknown substances by immobilizing a known substance on the ligand and searching for substances which interact with the ligand. Furthermore, by immobilizing a substance having unknown properties on the ligand and comprehensively searching for known substances which interact with the immobilized substance, it becomes possible to estimate the properties of the unknown substance in a simple and easy manner and with certainty.

By the term “recovery” as used herein, it is meant that an arbitrary biological substance is collected from a sample. The term “purification” means that an arbitrary biological substance is collected or recovered from a sample so as to increase the purity and concentration thereof. By “elution”, it is meant that one or one or more arbitrary biological substances are separated one by one from a sample using a carrier. “Separation” means parting a mixture into a fraction containing a certain component and a fraction not containing that component. The “container” or “vessel” so referred to herein is used to place the particles, i.e. the carrier, of the invention therein and includes test tubes, columns, tubes, and micro centrifuge tubes, among others.

More Details Of Invention

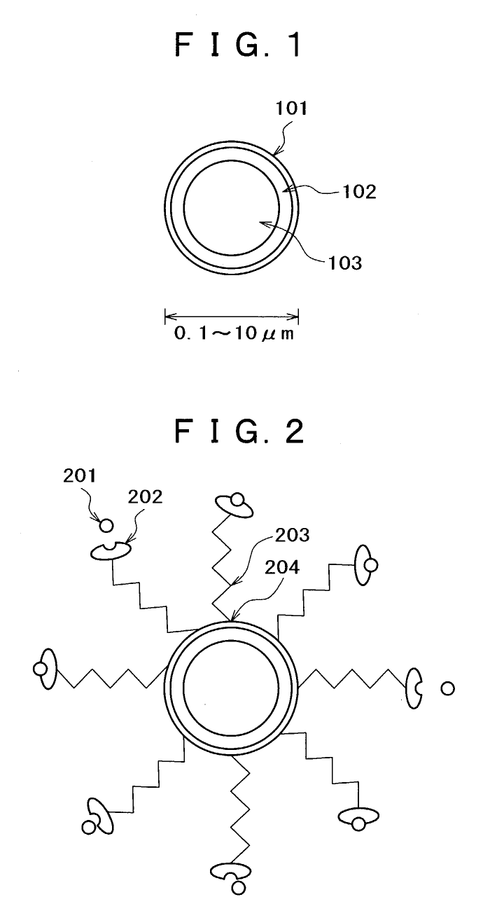

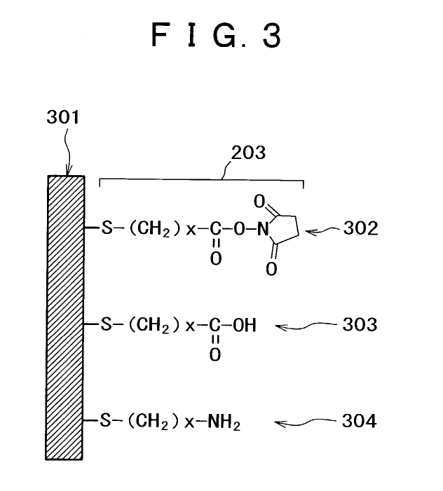

A method of spacer binding is described. Particles based on a polymer material derived from an ethylenically unsaturated group-containing monomer, which were uniform in size and sufficient in mechanical strength, were prepared according to the method described in JP-A No. 166228/2002 (Patent Document 2). The whole surface of the particles was covered with gold; thus, it became possible to provide the particles with electric conductivity and utilize the whole surface thereof for spacer binding, for instance. The structure of such a particle is schematically shown in FIG. 1 (101: layer of noble metal, 102: magnetic material, 103: polymer). The particles to be used are not smaller than 0.1 μm but not larger than 10 μm in diameter. The reason why the particles should have such a diameter is as follows: when the diameter is smaller than 0.1 μm, the particles are so small that the experimental procedure becomes difficult to carry out and, when it is greater than 10 μm, the procedure is restricted from the apparatus viewpoint in the subsequent analysis using a mass spectrometer, for instance. In practice, the particles, which had a diameter of 3 μm, were used. Then, as shown in FIG. 2 (201: biomoleculer species, 202: ligand, 203: spacer, 204: surface of the noble metal), a spacer was bound to the particle surface by the following treatment. Thus, 100 mg of the particles were placed in a 1.5-ml micro centrifuge tube, 1 ml of 37% aqueous hydrogen peroxide was added, the mixture was stirred vigorously and then centrifuged, and the supernatant hydrogen peroxide solution was removed. Washing was effected by adding 1 ml of ethanol and, after stirring and centrifugation, removing the supernatant. This washing procedure was repeated several times. After sufficient drying under vacuum, 1.5 ml of a 100 μmol/l solution of dithiobis(succinimidyl propanoate) in ethanol was added, and the mixture was stirred gently at room temperature, followed by centrifugation. The supernatant was removed. Washing was effected by adding 1.5 ml of ethanol and, after stirring and centrifugation, removing the supernatant. This washing procedure was repeated several times. Thereafter, the particles were sufficiently dried under vacuum. For binding a longer spacer, dithiobis(succinimidyl undecanoate) may be used in lieu of dithiobis(succinimidyl propanoate). While the concentration used in this example was 100 μmol/l, a solution within the concentration range of 10 μmol/l to 100 mol/l may be used. Certain typical spacers capable of binding to gold particles are shown in FIG. 3 (301: surface of the noble metal, 302: alkanethiol containing hydroxyl group, 303: alkanethiol containing carboxyl group, 304: alkanethiol containing amino group).

Ligand binding to the carrier is now described. The above spacer-bound particles were placed in a test tube, and the surface thereof was washed lightly with 1 mM HCl. Then, a ligand solution diluted to an appropriate level with a binding solution (200 mM NaHCO₃, 500 mM NaCl; pH 8.3) was added, and the mixture was shaken at room temperature for 30 minutes. The supernatant was removed by magnetic separation, the particles were washed with washing solution A (500 mM monoethanolamine, 500 mM NaCl; pH 8.3), then with washing solution B (100 mM sodium acetate, 500 mM NaCl; pH 4.0), and again with washing solution A. Then, washing solution A was added, and the mixture was shaken at room temperature for 60 minutes to effect blocking. The supernatant was completely removed by magnetic separation, and the particles were washed with washing solution B. Then, washing solution A was added and, after washing the particles, the supernatant was completely removed by magnetic separation. Then, phosphate-buffered saline (PBS) was added, and the mixture was stored at 4° C. until use in affinity experiments.

### EXAMPLE 1

An example of mouse IgG recovery from a hybridoma culture supernatant by immunoprecipitation using the carrier of the invention is shown below.

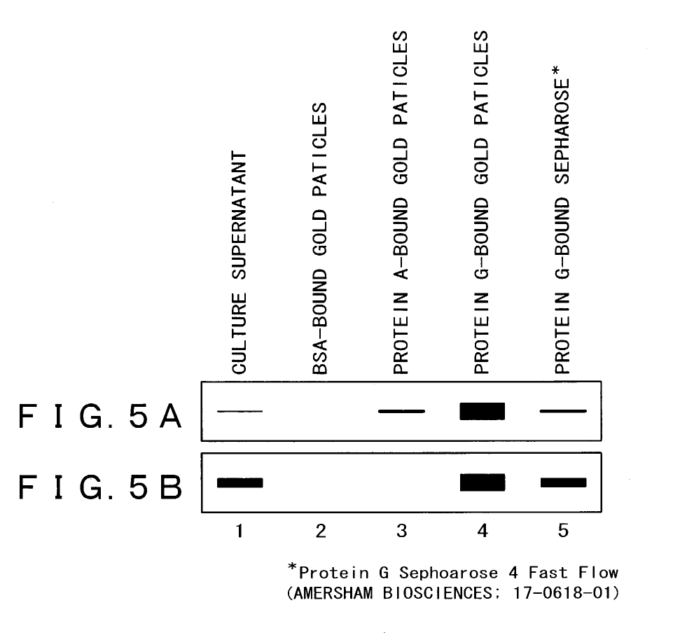

A mouse IgG-producing cloned hybridoma line was grown in RPM 1640 medium (IS Japan) containing 10% fetal calf serum (IS Japan) to a cell concentration of not less than 2×10⁶ cells/ml. The precipitate was removed by 5 minutes of centrifugation (Hitachi model 05PR-22 centrifuge) at 1000 rpm at room temperature, and the supernatant was used in the subsequent experiment. The particles with bovine serum albumin (BSA), protein A or protein G (all from Amersham Biosciences) bound thereto as a ligand were placed in a test tube, the hybridoma culture supernatant was added, and the reaction was carried out with slow stirring at 4° C. for 120 minutes. The supernatant was removed by magnetic separation, and the particles were washed with not less than 5 portions of a Tris solution (TBS) containing a final concentration of 0.05% of Tween 20. Then, a 0.1 M glycine solution (pH 3.0) was added to dissociate mouse IgG from the ligand. The pH of the mouse IgG-containing glycine solution was adjusted to about 7.0 with a 1 M Tris solution having a pH of 9.0. And a sample solution for SDS-polyacrylamide gel electrophoresis (SDS-PAGE) (62.5 mM Tris-HCl, 10% glycerol, 5% 2-mercaptoethanol, 2.5% SDS, 0.00125% bromophenol blue, pH 6.8) was added to the solution (FIG. 5A) or above (FIG. 5B) followed by 5 minutes of heating at 95° C.

Using these samples, SDS-PAGE was performed and, after transfer to a nitrocellulose membrane (Atoh) and labeling with a peroxidase-modified anti-mouse IgG antibody, visualization was effected by causing the emission of light with a chemiluminescence reagent (Pierce) and exposure of an X ray film to the light. As a result, when the particles with protein G, which is said to have high affinity for mouse IgG, immobilized thereon was used, a strong signal indicating the binding to IgG was observed, as shown in FIG. 5. It was thus revealed that the present invention is very useful in separating/isolating and concentrating biological substances from mixtures. FIG. 5 is a schematic representation of the results of purification of mouse IgG from a hybridoma culture supernatant using protein G immobilized on a carrier for biomolecule purification according to the present invention. FIG. 5A shows the results of western blotting of eluted fractions with a glycine solution and labeled with an anti-mouse IgG antibody. FIG. 5B shows the results of eluted fractions with an SDS-containing solution, of IgG remaining on the particles after the above glycine solution elution.

### EXAMPLE 2

An example of protein complex proteasome recovery from HeLa cells by immunoprecipitation using the carrier of the invention is shown below.

Cells of the human cervical carcinoma-derived cell line HeLa were sown in a dish (Falcon) having a diameter of 100 mm and containing DMEM [Dulbecco's modified Eagle's medium (Sigma), 100 μg/ml kanamycin (Gibco), 10% fetal calf serum (Sigma), MEM NEAA (MEM non-essential amino acids solution) (Gibco)] and, after attainment of about 70% confluent growth, cells were treated with trypsin and recovered. Thereto was added 200 μl of a cytolytic solution (20 mM HEPES, 150 mM NaCl, 1 mM EDTA, 1.0% Triton X-100, 0.5% deoxycholate, 0.1% SDS; pH 7.5) for suspending the cells. After 20 minutes of standing on ice, the suspension was centrifuged (15,000 rpm×30 minutes, 4° C.), and the supernatant was used as a disrupted cell solution in the subsequent experiment. Two rabbit-derived anti-proteasome antibodies, namely PA-969 which recognizes the S7 subunit of the proteasome 19S complex, and PA-970 which recognizes the S8 subunit of the proteasome 19S complex (both from Affinity Bioreagents), were used as the ligands, and each was immobilized on the particles by the method mentioned hereinabove. Thereto was added the disrupted cell solution, and the mixture was stirred at 4° C. for 1 hour using a micro tube mixer to promote the binding between the antibody and the proteasome in the disrupted cell solution. The supernatant was then completely removed by magnetic separation, and the particles were washed with three portions of the cytolytic solution. Finally, the supernatant was removed by centrifugation (15,000 rpm×1 minute, 4° C.), the sample solution for SDS-PAGE (62.5 mM Tris-HCl, 10% glycerol, 5% 2-mercaptoethanol, 2.5% SDS, 0.00125% bromophenol blue, pH 6.8) was added, and the mixture was heated at 90° C. for 3 minutes for eluting the protein bound to the particles therefrom. After ice cooling of the sample mixture, the beads (particles) were removed by centrifugation (15,000 rpm×5 minutes), and the sample thus obtained was subjected to western blotting and the results were analyzed.

In the western blotting, the PA-964 antibody (Affinity Bioreagents) recognizing the S2 subunit of the proteasome 19S complex was used as the primary antibody, and an alkaline phosphatase-bound anti-rabbit IgG antibody (Promega) as the secondary antibody after 5000-fold dilution. If proteins A, B and C form a protein complex, the protein C will be precipitated together with A and B on the occasion of immunoprecipitation with an anti-A antibody and an anti-B antibody, hence, when the thus-obtained immunoprecipitate fraction is subjected to western blotting using an anti-C antibody as the primary antibody, a signal of the protein C will be detected in the fraction resulting from immunoprecipitation using the anti-A or anti-B antibody. Based on this working hypothesis, experiments were carried out. As a result, when the protein fractions obtained by using the PA-969 and PA-970-antibodies were subjected to western blotting using PA-964 as the primary antibody, signals were obtained, suggesting that the S2 subunit of the 19S complex, which is recognizable by PA-964, forms the protein complex together with the S7 and S8 subunits of the 19S complex, which are recognizable by the other antibodies. Like in Example 1, the use of the carrier of the invention makes it possible to recover aggregates of functional molecules, such as protein complexes, in a simple and easy manner and rapidly through immobilization of such biological substances on appropriate ligands.

### EXAMPLE 3

An example of mouse IgG purification from the ascitic fluid by affinity column chromatography using the carrier of the invention is shown below.

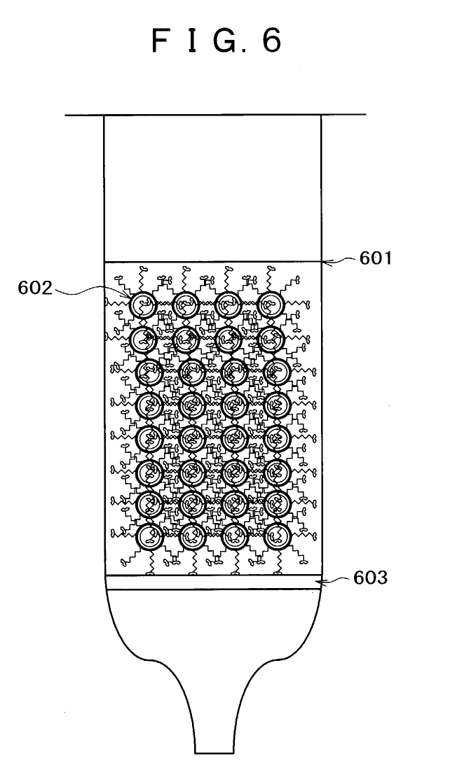

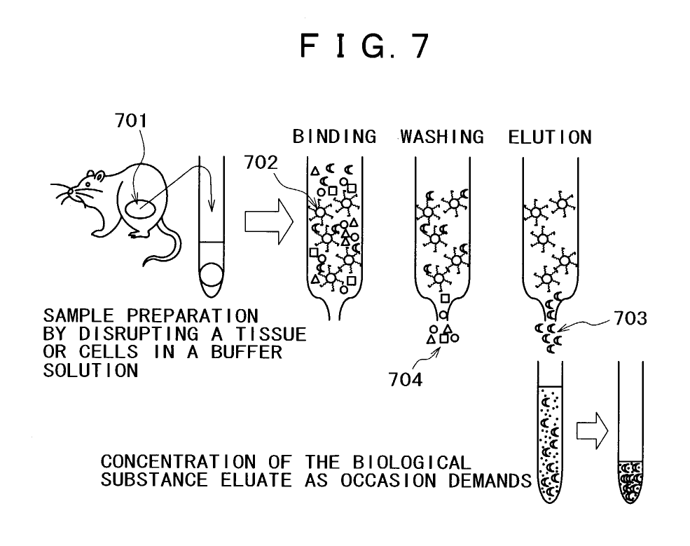

The carrier of the invention with protein G immobilized thereon as a ligand by the method mentioned above was packed in a column such as one shown in FIG. 6 (601: column, 602: carrier particles binding ligands, 603: stopper of particles), and affinity column chromatography was carried out. The procedure is schematically shown in FIG. 7 (701: tissue or cells, 702: carrier particles binding ligands, 703: specific desired biomolecule species, 704: contaminated non-specific biomolecule species). The mouse ascitic fluid was collected, and the IgG fraction was roughly purified by precipitation with ammonium sulfate and then transferred to a binding solution (200 mM sodium phosphate, pH 7.0) using a desalting column (PD-10; Amersham Biosciences). Then, particles occurring in the solution were removed by passing the solution through a 45-μm disk filter (Millipore) to give a sample. This sample was added, at a rate of 0.5 drop/second, to the column equilibrated in advance by feeding 3 to 5 volumes of the binding solution (flow rate: 1 drop/second). Then, 5 to 10 volumes of the binding solution were fed to wash out nonspecifically bound components. IgG was eluted by feeding 5 volumes of an eluting solution (0.1 M glycine-HCl, pH 3.0) (flow rate: 1 drop/second). For preventing the eluate from denaturating, a neutralizing solution (1.0 M Tris-HCl, pH 9.0) was immediately added thereto to adjust the pH to around neutrality. The purified IgG fraction thus obtained was subjected to SDS-PAGE and western blotting. As a result, it was revealed that mouse IgG had been purified and that the present invention is useful in purifying biological substances from mixtures.

### EXAMPLE 4

An example of the combined use of the method of purification using the carrier of the invention and the application of a voltage is shown below.

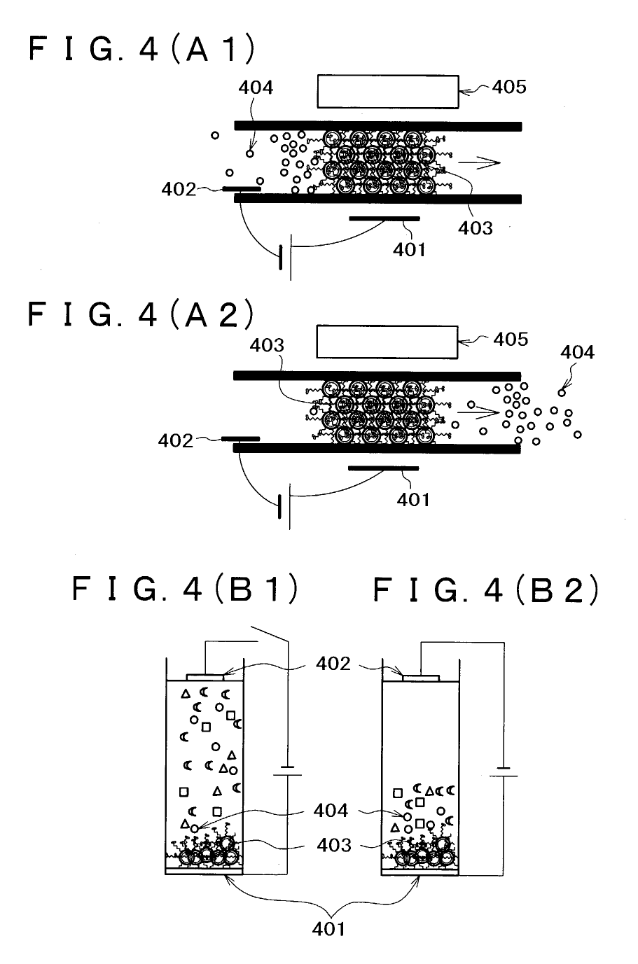

The efficiency of binding between a ligand and a biological substance can be increased when a voltage is applied to the carrier of the invention. The principle is schematically shown in FIG. 4 (401: electrode 1, 402: electrode 2, 403: carrier particles binding ligands, 404: desired biomolecule species, 405: magnetic fields). Thus, the carrier of the invention with a molecular species, which is capable of binding to a desired protein molecule species, immobilized thereon is introduced into a crude cell extract containing the desired protein molecule species. The particles constituting the carrier are placed in a state such that they are in contact with one another in the crude cell extract. The particles are caused to move under the influence of gravity of a magnetic field, for instance. FIGS. 4A-1 and 4A-2 schematically show the case of movement under the influence of a magnetic field, and FIGS. 4B-1 and 4B-2 the case of movement under the influence of gravity. A voltage is applied between an electrode 1 (FIG. 4, 401), disposed externally along one side of a vessel and another electrode 2 (FIG. 4, 402) separated from the former by the vessel wall. And biomolecule species are disposed at a certain place in the opposite direction to the movement of the particles within the extract; the voltage is thus applied to the extract. As a result, the biological substance is electrically caused to migrate between the electrodes and thus guided to the vicinity of the particles and collected, so that the rate of interaction between the ligand bound to the particles and the biological substance increases. For example, when the crude cell extract and carrier are placed in a 1.5-ml micro centrifuge tube and allowed to stand, the carrier spontaneously settles on the bottom of the tube and takes a state of contact with one another. When an electrode is disposed in advance on the bottom of the micro centrifuge tube, the carrier partly comes in contact with the electrode while maintaining the contact among the particles. The other electrode is brought into contact with the liquid surface in the tube, and a voltage of 8 V/cm (or a voltage of from 1 V/cm to 20 V/cm) is applied so that the bottom may serve as a negative electrode and the liquid surface as a positive electrode. In this example, the binding solution, eluting solution and so forth are selected according to the biological substance species to be bound to the ligand. The direction of electrodes can also be changed arbitrarily according to the sample.

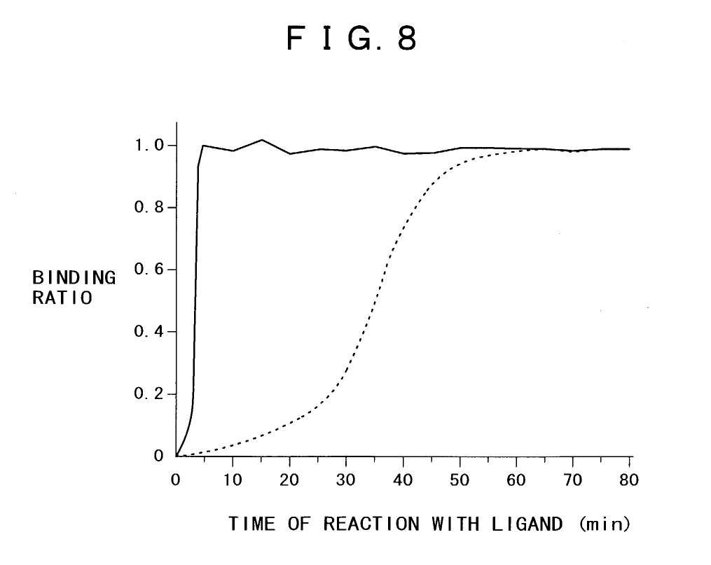

As shown in FIG. 8, the efficiency of binding between the ligand and biological substance, which binding requires 60 minutes or a longer period in the conventional art, can be increased by voltage application for collecting the biological substance in the vicinity of the particles. The dotted line graph shows results without application of an electric field. The solid line graph shows results with application of an electric field using devices of the present invention. Accordingly, the binding is complete in 3 to 5 minutes when the device of the invention is used. The efficacy thereof was thus proved.

In FIG. 8, a mixture of the ligand and biological substance was allowed to stand on ice for a specified period of time. The ordinate denotes the binding ratio with the binding obtained after standing until arrival of the reaction at a point of saturation being taken as 1. The abscissa denotes the time of standing.

### EXAMPLE 5

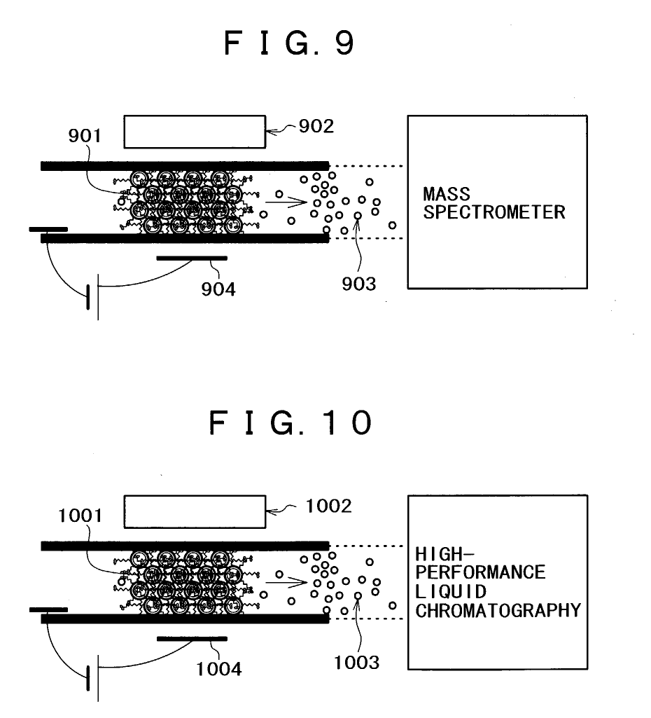

An example in which the carrier of the invention and the method described in Example 4 are applied to a system for analyzing biological substances in which a mass spectrometer is used in the analyzing section is shown in the following. As shown in FIG. 9 (901: carrier particles binding ligands, 902: magnetic field, 903: desired biomolecule species, 904: electrode), the carrier is packed in a column or a tube (not shown) having a small inside diameter as disposed just in front of and directly connected to a mass spectrometer, and the column or tube inside is equilibrated with a solution adapted to the mass spectrometer. Note that FIG. 9 shows the carrier packed in a column, as opposed to a tube, for explanatory purposes. However, the invention is not so limited and a tube may used in place of a column.

A solution containing the substance to be bound to the ligand is then fed, and a voltage is applied by the method shown in Example 4 to induce the formation of the ligand- and biological substance-bound particles. The ligand- and biological substance-bound particles are then transferred to the sample inlet to the mass spectrometer by means of a magnetic field formed by an electromagnet or permanent magnet, for instance, and the biological substance is eluted and separated just in front of the spectrometer. In this manner, the concentration of the biological substance to be introduced into the mass spectrometer can be increased and high levels of measurement sensitivity can be obtained. Even when the concentration of the biological substance to be assayed is very low, hence below the detection limit of mass spectrometry in the prior art, the present invention can detect or assay such substance and thus solve the problem just mentioned above.

### EXAMPLE 6

An example in which the carrier of the invention and the method described in Example 4 are applied to a system for analyzing biological substances in which the analyzing section comprises a high-performance liquid column chromatograph is shown in the following. As shown in FIG. 10 (1001: carrier particles binding ligands, 1002: magnetic field, 1003: desired biomolecule species, 1004: electrode), the carrier is packed in a column or a tube having a small inside diameter as disposed just in front of and directly connected to a high-performance liquid column chromatograph, and the column or tube inside is equilibrated with a solution adapted to the apparatus. Then, a solution containing the substance to be bound to the ligand is fed, and a voltage is applied by the method shown in Example 4 to induce the formation of the ligand- and biological substance-bound particles. The ligand- and biological substance-bound particles are then transferred to the sample inlet by means of a magnetic field formed by an electromagnet or permanent magnet, for instance, and the biological substance is eluted and separated just in front of the apparatus. In this manner, the concentration of the biological substance to be subjected to high-performance liquid column chromatography can be increased and high levels of separability and measurement sensitivity can be obtained. In the prior art, specific biological substances contained in respective fractions to be separated by high-performance liquid column chromatography are often diluted, and their inactivation has been a problem in certain instances. On the contrary, such problems can be solved by applying the method shown in Example 4 after separation of the biological substance to thereby adjust the concentration thereof.

### EXAMPLE 7

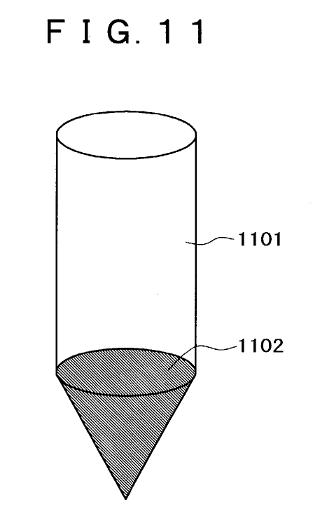

A further example of the present invention is shown in FIG. 11 (1101: tubing, 1102: gold coated surface). The inside bottom of a reaction test tube, for example a 1.5-ml micro centrifuge tube made of polystyrene or polypropylene, is partly covered with gold, and a spacer and a ligand are immobilized on the surface of the gold by the method mentioned hereinabove. Thereto was added dropwise a mixture containing a specific biological substance and, after a certain period of standing, the solution is removed by pipetting. Dropwise addition of a buffer solution followed by pipetting is repeated several times to thereby sufficiently wash out nonspecifically adsorbed substances, and the specific biological substance alone is separated and recovered.

When other various particles are used as specific adsorbent carriers, centrifugation and/or magnetic separation is indispensable throughout the working process. When the method according to the invention is employed, such procedure is not indispensable. The method is simple and easy to conduct and is also effective in reducing the working time and avoiding sample losses. It is very effective in handling trace amounts of samples, which have been difficult to handle in the prior art. The objects of the present invention can also be accomplished by the following constitutions. 1. A biological substance purification kit which comprises a container or vessel having an inside wall partly covered with a noble metal, with the noble metal surface being surface-treated for enabling binding of a capturing molecule species for binding a biomolecular species. 2. A biological substance purification kit as mentioned above in which the noble metal surface has the capturing molecule species immobilized thereon for capturing the biomolecular species.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method of purifying biological substances, the method comprising:
preparing a carrier species having a surface covered with a noble metal, wherein a surface of the noble metal is surface-treated for binding a capturing molecule species to a biomolecular species;
binding the capturing molecule species to the surface of the noble metal to create one or more carriers;
feeding a sample containing the biomolecular species to a container containing the one or more carriers;
causing the biomolecular species to bind to the capturing molecule species;
feeding a solution for one of dissociating or eluting the biomolecular species bound to the capturing molecule species on the one or more the carriers therefrom; and
recovering the biomolecular species.

2. The method of claim 1, wherein the step of causing the biomolecular species to bind to the capturing molecule species comprises applying a voltage to the sample to guide the biomolecular species to the vicinity of the carriers.

3. The method of claim 1, wherein the one or more carriers contain magnetic material, the method further comprising applying a magnetic field to transfer the one or more carriers, wherein the step of recovering the biomolecular species comprises feeding a solution to transferred carriers to recover the biomolecular species, wherein the solution is configured to dissociate or to elute the biomolecular species bound to the capturing molecule species.

4. A kit for biological substance purification, the kit comprising one or more carriers, each covered on substantially a whole surface of each carrier with a noble metal, wherein a surface of the noble metal is surface-treated in order to bind a capturing molecule species to a biomolecular species of each of the one or more species, wherein the capturing molecule species is configured to recover the biomolecular species.

5. The kit of claim 4, wherein the surface of the noble metal has the capturing molecule species for capturing the biomolecular species as bound thereto.

6. The kit of claim 4, wherein the noble metal is selected from the group consisting of gold, platinum, silver and copper.

7. The kit of claim 4, wherein the one or more carriers are magnetic material-containing carriers.

8. The kit of claim 4, wherein the carriers have a diameter of between about 0.1 μm and about 10 μm.

9. The kit of claim 4, wherein the carriers have a specific gravity of not less than 1.0.

10. The kit of claim 4, wherein the surface treatment comprises thiolation of the surface of the noble metal.

11. The kit of claim 4, wherein the surface treatment comprises binding of an active group-terminated alkanethiol to the surface of the noble metal through addition of thiol group to the surface.

12. The kit of claim 7, each of the magnetic material-containing carriers comprising:
a layered structure including a central portion made of a polymer;
a layer surrounding the polymer and including the magnetic material; and
a layer of the noble metal externally covering the magnetic material layer.

13. A system for analyzing a biological substance, the system comprising:
a column or tube containing one or more carriers, wherein each carrier contains a magnetic material and is covered by a noble metal, wherein a capturing molecule species is bound to a surface of each carrier;
an analyzing section connected to the column or tube;
a transfer device for transferring the one or more carriers within the column or tube; and
a voltage application device configured to guide a biomolecular species to a vicinity of the one or more carriers, wherein the column or tube is fed with a sample containing the biomolecular species for binding of the biomolecular species to the capturing molecule species, wherein the column or tube is fed with a solution for dissociation or elution of the biomolecular species bound to the capturing molecule species on the one or more carriers therefrom to thereby recover the biomolecular species, wherein the analyzing section is configured to analyze the biomolecular species that is recovered.

14. The kit of claim 13, wherein the analyzing section comprises a mass spectrometer for analyzing the biomolecular species that is recovered.

15. The system of claim 13, wherein the analyzing section comprises a high-performance liquid chromatograph configured to analyze the biomolecular species that is recovered.

16. The kit of claim 13, wherein the transfer device is a magnetic field application device disposed externally to the column or tube.

17. The system of claim 13, the voltage application device comprising:
a first electrode disposed externally to a container containing the one or more carriers, wherein the first electrode is in indirect contact with one or more carriers;
a second electrode in contact with the sample in the column or tube; and
a power source configured to apply voltage between the first electrode and the second electrode.
